(19) United States
(12) Patent Application Publication
(10) Pub. No.: US 2013/0218582 A1
(43) Pub. Date: Aug. 22, 2013

(54) TELEMEDICINE SYSTEM FOR IMD PATIENTS USING AUDIO/VIDEO DATA

(71) Applicant: John LaLonde, Lake Elmo, MN (US)

(72) Inventor: John LaLonde, Lake Elmo, MN (US)

(73) Assignee: CARDIAC PACEMAKERS, INC., St. Paul, MN (US)

(21) Appl. No.: 13/591,474

(22) Filed: Aug. 22, 2012

Related U.S. Application Data
(60) Provisional application No. 61/557,208, filed on Nov. 8, 2011.

(51) Int. Cl. A61B 5/00; A61B 5/0205

(52) U.S. Cl. CPC A61B 5/0024; A61B 5/0205
USPC 705/2; 600/324; 600/301

(57) ABSTRACT

A method and system for assisting a remote implantable medical device (IMD) clinician with evaluation of a patient having an IMD. The method includes capturing audio/video data of the patient while the patient performs a first evaluation protocol at a first location and performing a pattern recognition analysis of the audio/video data of the patient. The analysis includes comparing specific portions of the audio/video data to like portions of previous audio/video data of the patient that were recorded while the patient performed the first evaluation protocol. Yet another step is providing a report of any changes identified during the pattern recognition analysis.

This application claims the benefit of U.S. Provisional Application No. 61/557,208, filed Nov. 8, 2011, the contents of which are herein incorporated by reference.

### FIELD OF THE INVENTION

The invention relates to the care and evaluation of patients having implantable medical devices (IMDs) such as cardiac rhythm management devices. More particularly, the invention relates to methods and systems for remotely evaluating patients having IMDs.

### BACKGROUND OF THE INVENTION

Many different types of medical devices are implanted within patients to provide medical therapy. One type of an implanted medical device is a cardiac rhythm management device, such as a pacemaker or implantable defibrillator. Cardiac rhythm management (CRM) devices are used to provide medical therapy to patients who have a disorder related to cardiac rhythm, such as bradycardia or tachycardia.

Systems to provide remote monitoring of IMDs have allowed clinicians to review reports from the IMDs and thereby receive detailed information about the patients. These systems have decreased the need for personal visits to a clinic office or physician location. However, there are benefits to personal visits with health care professionals in the form of visual and audio observation of the patient. Seeing a patient's face and hearing a patient's voice can provide important information about the patient's condition and overall health status. For example, for a patient with a CRM device, a puffy face may indicate edema which is a sign of cardiac decompensation. Other types of audio/video observation provide other types of important patient information. Clinicians would benefit from improved systems for providing and analyzing audio/video patient data.

### SUMMARY

In one embodiment, a method is described for assisting a remote implantable medical device (IMD) clinician with evaluation of a patient having an IMD. One step is capturing audio/video data of the patient while the patient performs a first evaluation protocol at a first location and performing a pattern recognition analysis of the audio/video data of the patient. The analysis includes comparing specific portions of the audio/video data to like portions of previous audio/video data of the patient that were recorded while the patient performed the first evaluation protocol. Yet another step is providing a report of any changes identified during the pattern recognition analysis.

In another embodiment, the steps include providing a patient interface device at a patient location configured to retrieve identity data and sensor data from an implantable medical device (IMD). Another step is providing a clinician interface device at a clinician location configured to be in remote, bi-directional audio/video communication with the patient interface device, where the patient interface device captures the audio/video data and where the report is provided to the clinical interface device.

In one embodiment, where the patient interface device is configured to program the IMD with IMD settings.

In another embodiment, the method further includes generating an alert if the changes identified during the pattern recognition analysis meet alert threshold requirements.

In another embodiment the report includes a heart failure decompensation status of the patient. In another embodiment, the report includes IMD sensor data.

In one embodiment, a system is for assisting a remote implantable medical device (IMD) clinician with evaluation of a patient having an IMD. The system includes a patient interface device configured to retrieve identity data and sensor data from an implantable medical device (IMD) at a first location and capture audio/video data of the patient while the patient performs a first evaluation protocol at the first location. The system also includes a clinician interface device configured to be in remote, bi-directional audio/video communication with the patient interface device. The system is configured to perform a pattern recognition analysis of the audio/video data of the patient, including comparing specific portions of the audio/video data to like portions of previous audio/video data of the patient that were recorded while the patient performed the first evaluation protocol. The system is configured to provide to the clinician interface device a report of any changes identified during the pattern recognition analysis.

The invention may be more completely understood by considering the detailed description of various embodiments of the invention that follows in connection with the accompanying drawings.

### BRIEF DESCRIPTION OF THE DRAWINGS

FIG. 1 is a schematic view of one embodiment of a system for assisting a clinician with evaluation of a patient having an IMD.

FIGS. 2 and 3 are perspective views of one embodiment of a patient interface device for use with the system of FIG. 1.

FIG. 4 is a flowchart showing a method for assisting a clinician with evaluation of a patient having an IMD.

FIG. 5 shows one example of a report on a pattern recognition analysis.

FIG. 6 is a schematic diagram of an implementation of the components of an interface device, in accordance with various embodiments.

FIG. 7 is a block diagram of an implantable medical device.

While the invention may be modified in many ways, specifics have been shown by way of example in the drawings and will be described in detail. It should be understood, however, that the intention is not to limit the invention to the particular embodiments described. On the contrary, the intention is to cover all modifications, equivalents, and alternatives falling within the scope and spirit of the invention as defined by the claims.

### DETAILED DESCRIPTION

### System Overview

The invention relates to the care and evaluation of patients having implantable medical devices (IMDs) such as cardiac rhythm management devices. More particularly, the invention relates to methods and systems for remotely evaluating patients having IMDs. Still more particularly, embodiments of the invention relate to a clinician efficiently and remotely evaluating patients with IMDs and performing useful pattern recognition analysis on audio/video data from those patients.

The term “audio/video” as used herein means audio or video or both. The term “clinician” as used herein means a person with medical experience or training including one or more of training on the function and programming of IMDs, medical training and medical evaluation protocol training Examples of a clinician are a medical doctor, a nurse, a medical technician, an electro-physiologist, a cardiologist, a cardiology technician, a cardiology nurse, or a surgeon. A clinician may undergo special training or qualification related to a particular IMD, a particular health condition or a particular evaluation protocol.

As used herein, the term “pattern recognition” means the analysis and deterministic assignment of some sort of output value or label to a given input value or input data, according to a heuristic algorithm. A telemedicine system as described herein can provide a remote clinician with audio/video data from a patient with an IMD, can perform pattern recognition analysis of the audio/video data in order to assist the clinician, and can provide one or more outputs from the pattern recognition analysis that provide patient information to the clinician. For example, the system can record audio data of the patient's speech while the patient repeats specific words according to an evaluation protocol. The system can then compare audio data synchronized to similar audio data recorded at an earlier time. The system can determine if there has been any frequency shift in the patient's voice. The system can also quantify the frequency shift and can report to the clinician on whether a frequency shift to lower frequencies, if any, exceeds a threshold and may indicate that the patient is retaining fluid. In another example, the system can capture multiple images of the patient's hands or another body part while the patient holds the body part in a specified position. The system can then compare these images to similar images captured at an earlier time and determine if and can estimate by how much the area in an image or volume of body part has increased. The system can also report any increase to the clinician on an absolute basis or relative percentage and can report whether the increase exceeds a threshold and may indicate fluid retention or weight gain. Other examples of pattern recognition analysis will be further described herein. The system can also provide the audio/video data directly to the clinician for direct review.

The system is also capable of communicating with the patient's IMD and retrieving sensor readings and other data from the IMD. The system can report the pattern recognition analysis results to the clinician alongside relevant sensor data from the IMD. Efficient presentation of these different types of patient data assists a clinician with recognizing a change in the patient's health status and can also be used to trigger an alert in a patient management system.

The system includes a patient interface device which is designed to be at a first location which is the patient's location. The patient's location could be a medical clinic, assistance living center, cardiologist office, physician office, the patient's home or many other locations. The patient interface device is configured to retrieve identity data and sensor data from an implantable medical device (IMD), capture audio/video data of the patient while the patient performs at least one evaluation protocol and program the IMD with IMD settings, if needed.

The telemedicine system also includes a clinician interface device at a clinician location remote from the patient. The clinician interface device is configured to be in remote, bi-directional audio/video communication with the patient interface device. The clinician and patient can speak to and see each other in real time, even though they are in different locations. The clinician interface device provides video images and audio of the patient, and the patient interface device provides video images and audio of the clinician.

The telemedicine system is configured to perform a pattern recognition analysis of the audio/video data of the patient. During the pattern recognition analysis, the specific portions of the audio/video data are identified and then compared to like portions of previous audio/video data of the patient that were recorded while the patient performed the same evaluation protocol. The pattern recognition analysis may be performed by the patient interface device, by the clinician interface device, or by another computer system connected via the network. The telemedicine system is also capable of providing to the clinician interface device a report of any changes identified during the pattern recognition analysis. The report can include sensor data from the IMD, audio/video data, a description of the protocol and many other pieces of information. The system is also capable of providing alerts and recommending actions.

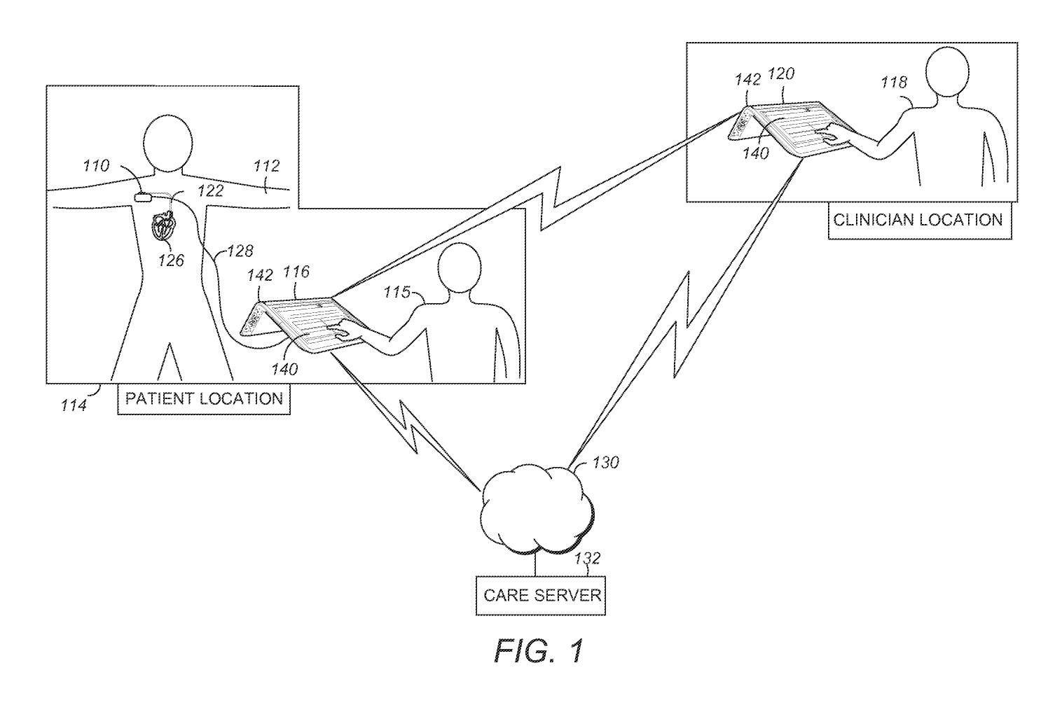

FIG. 1 is a schematic view of one embodiment of a telemedicine system of the present invention. In this system, an IMD 110 is shown implanted in a patient 112. The patient 112 is shown in a patient location 114, along with a health care professional 115. The health care professional uses a patient location interface device 116 to download data from the IMD and to communicate with a remote clinician 118. The remote clinician 118 is using a clinician interface device 120 to interact with the health care professional 115 and the patient 112, as well as to view IMD data, audio/video data and the results of pattern analysis recognition. It is also possible for the system to be used without the presence of a health care professional 115 at the patient location. In many situations, the patient can be directed to take the steps that would otherwise be taken by the health care professional. Further details about the various parts of the telemedicine system will be described herein.

### Patient Location Systems and Methods

In the example of FIG. 1, the patient 112 has a cardiac IMD 110 including cardiac leads 122 and located near the patient's heart 126. Examples of IMDs 110 include, without limitation, a cardiac device, a pacemaker, a defibrillator, a cardiac resynchronization therapy (CRT) device, an insulin pump, a loop recorder, a neuro-stimulator, a physiological sensor, a glucose meter or a combination of such devices. The system also includes the patient location interface device 116 or other external device that communicates wireless signals with the IMD 110, such as by using radio frequency (RF) or other telemetry signals. Alternatively or in addition, the patient interface device 116 communicates inductively with the IMD using a telemetry wand 128.

The patient interface device 116 is configured to be in remote, bi-directional communication with the clinician interface device 120, which is at a remote location. “Remote” is used to mean not in the same physical space, although it does not require a particular distance of separation. The communication link between the patient location interface device 116 and the clinician interface device 120 may be via phone lines, a wired network, the Internet 130, a pervasive wireless network or any other data connection. The patient interface device 116 can also be used when it is not in communication with an IMD device, but is only in communication with the clinician interface device 120. The patient interface device 116 can also be used when it is not in communication with the clinician interface device 120, but is only in communication with an IMD.

Generally, a pervasive wireless communication network is a communications network that can be used to directly communicate with a host computer without the need for a repeater device. A pervasive network includes those networks that are sufficiently prevalent or dispersed that an average person in the U.S. would be within range of interfacing with the network at some point during a normal daily routine. A pervasive wireless network typically has a relatively broad effective geographic span. There are many different usable pervasive wireless communication networks. One example is a wireless telephone network, such as a cellular telephone network. Other example embodiments of a pervasive wireless communication network include a wireless pager network, wireless wide area networks (WAN), such as those installed in certain public places like coffee shops, airports, schools, or municipalities, and wireless local area networks (LAN) including those following the standards set forth by the Institute for Electrical and Electronic Engineers (IEEE) in Standards 802.11(b) and (g).

In one embodiment, the patient interface device 116 can also communicate with an external storage media. The communication with the external storage media allows storage of IMD data, settings of the IMD, IMD settings that are entered using the patient interface device 116 and audio/video data of the patient. In the embodiment of FIG. 1, the external storage media is a part of a server 132. It is also possible for the external storage media to be a portable storage device, such as a flash drive or magnetic card. Although the term “server” will be used to describe the storage location in a number of embodiments described herein, including the embodiments of FIG. 1, it will be understood that it is also possible for the same data to be stored on an external storage media different than a server. In one embodiment, the patient interface device includes a local memory location. Audio/video data, IMD data and other data is stored in the local memory location in some embodiments. In another embodiment, some information is stored in a local memory location while some information is stored on an external storage media such as a server.

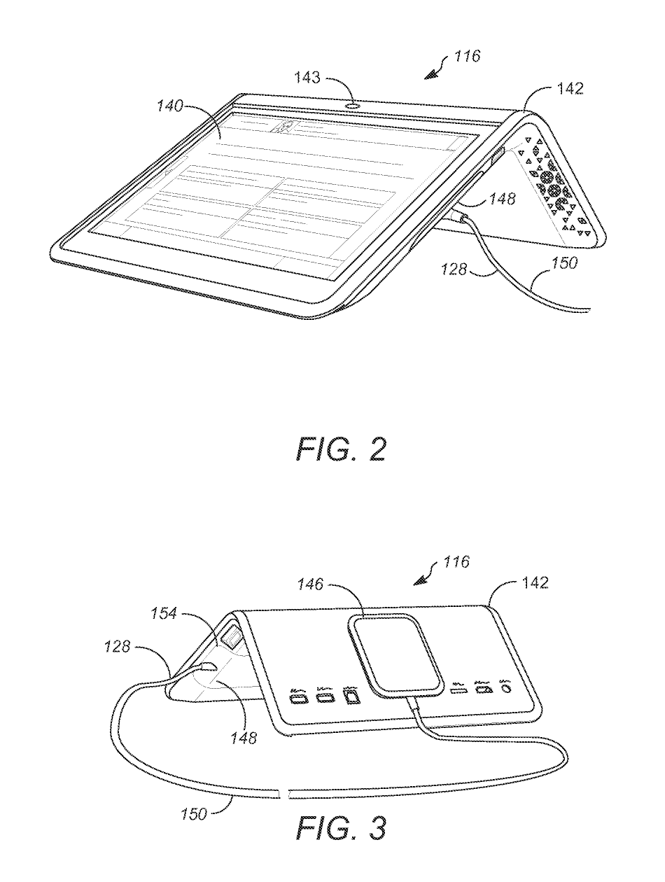

There are many options for the hardware of the patient interface device 116 and some of these options will be further described herein. FIGS. 2-3 illustrate two perspective views of one embodiment of a patient interface device 116. In one embodiment, the patient interface device 116 includes a touch screen 140, a wand 128, and a housing 142. The patient interface device 116 includes a camera 143, one or more speakers, and a microphone. In other embodiments, the patient interface device 116 includes other user input devices such as a keyboard and a mouse. In the patient location setting, a health care professional 115 can use the patient interface device to program IMDs, record data from IMDs, allow monitoring of the implanted device and upload information to a server 132. The health care professional 115 can also use the patient interface device to record and transmit audio/video data from the patient 112 for storage, evaluation by the remote clinician, evaluation by another health care professional, storage in a patient's electronic medical records or other purposes.

One configuration for a patient interface device is shown in FIGS. 1-3 as a tablet-style computer with a fixed, angled touch screen. But many other configurations are possible. For example, the patient interface device may take the form of an IMD programmer, a ruggedized laptop computer, a laptop computer, a desktop computer, a tablet computer, a smart phone or another configuration. The patient interface device 116 can also be referred to as a device, programmer, a user interface or an external interface device. In one embodiment, the patient interface device 116 is in communication with a server, which can be referred to as a care server 132. The communication link between the patient interface device 116 and the server 132 may be via phone lines, a wired network, the Internet 130, or any other data connection, such as a pervasive wireless network. The patient interface device 116 can also be used when it is not in communication with an IMD device, but is only in communication with the server 132.

In one embodiment, the patient interface device 116 is configured for portability by a user. For example, in one embodiment, the weight of the device 116 is less than three pounds. In another embodiment, the device 116 less than two pounds. In one embodiment, a power source of the device 116 is contained within the housing 142. The device 116 includes a touch screen 140 in one embodiment, which allows for both a graphical display and user input.

The device 116 includes a first communication module in the housing 142 configured to communicate with one or more of the clinician interface device 120, the server 132 and other locations using a first communication link. The first communication link between the device 116 and these other locations may be via a pervasive wireless network, conventional phone lines, the Internet 130, or any other data connection.

The device 116 further includes a device communication module 128, also referred to as a telemetry wand 128 in some embodiments, for communicating with the IMD 110 over a device communication link. Now referring to FIGS. 2-3, in one embodiment, the device communication module 128 includes an induction unit 146 on one end, an interface module 148 on the other end, and a flexible cord 150 connecting them. The induction unit 146 can be positioned over the IMD to enable inductive communication with the IMD. In an alternative embodiment, the device communication module uses radio frequency (RF) or other telemetry signals to communicate with the IMD.

In one embodiment, the interface module 148 is configured to be easily attached to and detached from the rest of the device 116. FIG. 3 illustrates the device communications module 128 attached to the rest of the device 116. The interface module 148 includes an interface connector structure 152 which reversibly connects to a mating connector structure (not shown) on the device 116. The connector structure is removable from and replaceable onto with the mating connector structure. The interface connector structure is capable of carrying digital, bi-directional communication between the device communication module 144 and the rest of the device. The interface connector structure is also capable of providing power to the device communication module 128 from the device 116. In one embodiment, a universal serial bus (USB) data transfer protocol is used. Other data transfer protocols and connector configurations could be used instead, such as an Ethernet connector, an IEE1394 interface (also known by the trade names of FireWire sold by Apple and Think sold by Sony) and a Thunderbolt interface. The interface connector structure is a USB connector with additional power connections in one embodiment.

In order to connect the interface module 148 to the patient interface device 116, the interface module 148 slides into a recess 154 on the side of the device 116. When the interface module 148 is fully engaged in the recess 154, the connector engages the mating connector of the programmer. In one embodiment, then the interface module 148 is fully engaged in the recess, the exterior of the interface module 148 is flush with an exterior surface of the device 116.

It is possible that the patient interface device 116 could be used with a device communication module 128 provided by a manufacturer different from the manufacturer of the patient interface device, but still be configured to attach to the device 116. Such an arrangement would allow the clinician and health care professional to use a single device 116 and user interface to conduct patient evaluations and make programming changes to IMDs from different manufacturers, thereby simplifying the equipment required to care for patients. In one embodiment shown in FIG. 3, the inductive unit 146 is configured to mechanically fit into and be held by a cradle structure on a back side of the device 116, for the convenience of the user of the device.

One embodiment of a patient interface unit is described as a programmer 116 in commonly-owned provisional patent application No. 61/529,592 which is hereby incorporated herein by reference in its entirety.

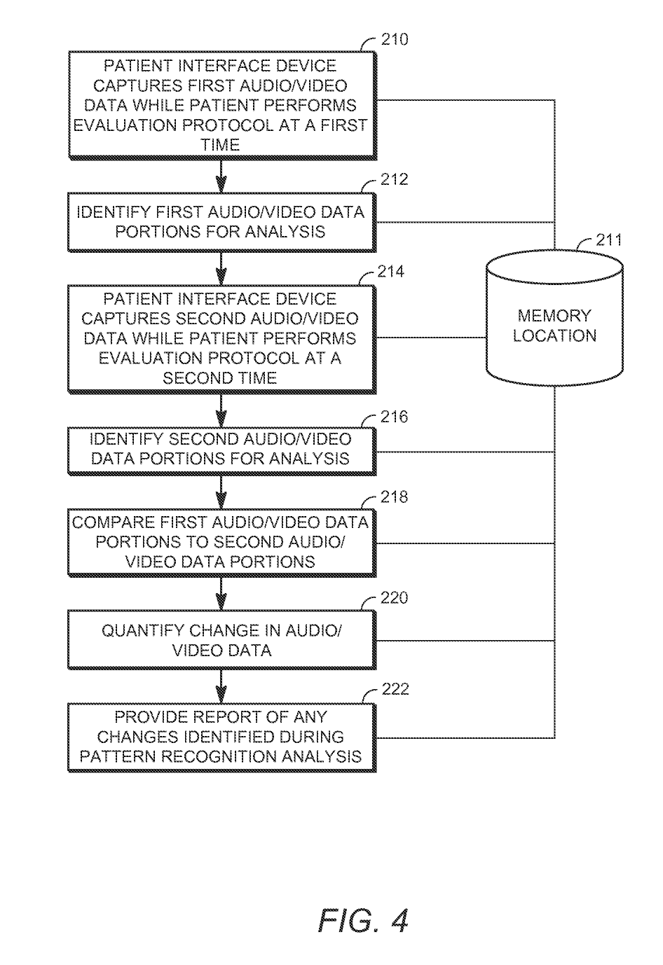

FIG. 4 shows one embodiment of a method for assisting a clinician with evaluation of a patient, where the patient has an implantable medical device. The method utilizes a patient interface device at the patient location and a clinician interface device at the clinician location. In step 210, the patient performs an evaluation protocol and the patient interface device captures first audio/video data of the patient performing the evaluation protocol. Step 210 takes place at a first time, and results in the first audio/video data being stored in a memory location 211. As a next step 212, the system identifies specific portions of the first audio/video data for analysis. In one example the first data is audio data of the patient speaking a string of words. The system will identify the location within the audio data of the beginning of the first word and the location of the end of the last word. The portions in between will be used for pattern recognition analysis. The first audio/video portions identified in step 212 will also be stored in the memory location 211.

At a second time, a second piece of audio/video data is captured in step 214 while the patient performs the same protocol as was performed at the first time. Then second audio/video data portions are identified for analysis, similar to the first audio/video data portions discussed above. The steps of identifying the specific portions of the audio/video data for analysis help to ensure that the system is making a useful comparison of like or similar data gathered at different times, so that the system will be able to accurately identify changes in the patient. The second piece of audio/video data and the location of the specific portions for analysis within the second audio/video data are also stored in the memory location.

At step 218, the system compares the first audio/video data portions to the second audio/video data portions. At step 220, the system quantifies the change in the audio/video data. In one example, the system determines a quantity of a frequency shift in the patient's voice. In another example, the system determines a quantity of an increase in the area of the patient's body part on an image, such as the patient's hands.

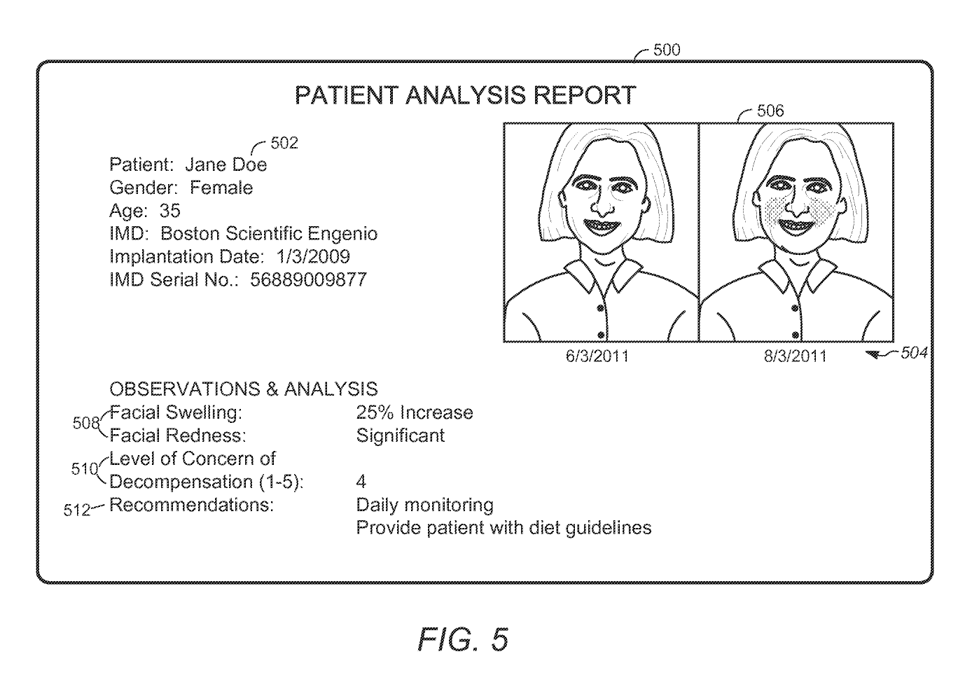

At step 222, the system provides a report of any changes identified during the pattern recognition analysis. The output of the comparison and the report are also stored in the memory location 211. An example of a report 500 is shown in FIG. 5. Components of the report in one embodiment include an identification of the patient 502, an identification of the time and location of the recording of the first and second audio/video data 504, one or more images of the patient 506, and a quantification of a change 508 of one or more aspects of the patient's audio/video data. Further possible components of a report include a description of the evaluation protocol and a description of the pattern recognition analysis that produced the quantified output. The results of a comparison of the quantification to a threshold can be used to produce an index or indicator of concern 510 for the patient. This index or indicator of concern can lead to a clinical decision by the clinician in some cases. For example, a high level of concern may lead the clinician to a decision that the patient is experiencing decompensation and should be hospitalized for immediate treatment. In another example, an index may indicate to the clinician that the patient has weight gain. The weight gain can be reported to the clinician and also to the patient's electronic medical record and a patient care server. The clinician may utilize the patient interface device to provide a diet for the patient to follow closely to attempt to slow or reverse the weight gain. The output may cause an alert to be sent to a patient care system. In the example report of FIG. 5, recommendations 512 are provided to the clinician to provide daily monitoring and diet guidelines.

In various embodiments, the report can include sensor data from the IMD, other sensor data, audio/video data, a description of the protocol and many other pieces of information. Examples of sensor data that can be included in a report include blood pressure, changes in blood pressure, pulse oximetry, changes in pulse oximetry, the patient's weight and changes in the patient's weight. Examples of IMD sensor data that can be included on the report include respiration rate, changes in respiration rate, recent cardiac pacing, cardiac pacing changes, transthoracic impedance and changes in transthoracic impedance. The report may include one or more of these types of sensor data, and may include them in any combination.

Another step that can be taken by the patient interface device in some embodiments is to receive sensor data, such as temperature, blood pressure, pulse oximetry and weight. The sensor data can be input by a health care professional. The patient interface device can also receive sensor data directly from a sensor, such as by electronic communication with a blood pressure cuff, a scale or a pulse oximetry sensor.

Additional steps that are taken by the patient interface device in some embodiments include interrogating the IMD to determine its status and download diagnostic information, therapeutic information and IMD sensor data. An additional step that can be taken by the patient interface device is to provide instructions to the patient for the evaluation protocol. The instructions about the evaluation protocol are formulated to assist the professional and the patient with creating useful audio/video data for recording by the patient interface device. In one example, the instructions are provided on the display screen. In one example, the audio instructions are played by the patient interface device. In another example, the instructions are both shown on the display screen and audio instructions are played. In one example, the instructions are provided on the display of the patient interface device, and are read by the health care professional to the patient. The patient may also view the instructions during all or part of the protocol. The patient can read the instructions independently in some embodiments. In another example, the instructions are provided verbally by the remote clinician who is visible to the patient on the touch screen and can be heard through the patient interface device.

Another step that can be taken using the patient interface device is identification of the patient. Identity data is stored in the IMD in one embodiment, such as an identification number, that uniquely identifies the patient, the particular IMD or both. In one embodiment, the unique identity data stored on the IMD is a device serial number. To retrieve the identity data, the patient interface device 116 can communicate with the IMD 110 inductively, using the device communication module 144, and retrieve the identity data from the IMD 110. In some embodiments, the induction unit 146 is positioned over the IMD 110 to facilitate inductive communication. Alternatively, the camera of the device 116 can be used to confirm the patient's identity using facial recognition software or other biometric data. It is also possible to use a portable storage device, such as a magnetic card storing identity information, to provide the identity information to the device. It is also possible for the patient to provide a name and a password to the device to verify identity.

### Evaluation Protocol Examples

In order to provide input for the pattern recognition analysis, the evaluation protocol is performed at an initial or first time and then at a later time when it is desirable to know if and how the patient has changed. The audio/video data collected at the second, later time is compared to the audio/video data collected at the first, earlier time (also referred to as the previous time). Many different evaluation protocols are possible for collecting the audio/video data. In each protocol, the patient may be positioned at a specific distance from the patient interface device and then be asked to take certain actions.

In one example of an evaluation protocol, the goal is to determine if the patient's voice has shifted in frequency compared to a previous recording, which may indicate edema or weight gain. In this example, the audio/video data is audio data. In such an example, the patient interface device can provide instructions for the patient to position himself or herself a specific distance from the patient interface device, so that the patient's mouth is a specific distance from the microphone. The instructions may direct the patient to say a specific set of words at a specific cadence, and then remain quiet for a period of time, such as five seconds, and then speak several specific words in the specified order at a specific cadence. The instructions may indicate that the patient or the health care professional can touch a button on the touch screen when the patient has finished saying the list of words. The system can provide the patient direct feedback on if the evaluation protocol was performed appropriately. A timer counting down the period of silence and the list of specific words can be displayed on the touch screen.

The system will identify the location within the audio data of the beginning of the first word and the location of the end of the last word. The portions in between will be used for pattern recognition analysis and stored in the memory location. Using spectrum analysis techniques, the pattern recognition analysis will determine which audio frequencies are present in the first and second audio portions. Then the system will determine whether there has been any shift in overall frequency in the patient's voice from the first time to the second time. One output of the analysis can be a statement of the quantification of the change in frequency shift, if any. For example, the report may state that the patient's voice has shifted by 10 Hertz or 20 Hertz or that there has been no shift. The system may compare the frequency shift to a threshold, and may indicate if the patient's frequency shift exceeds the threshold.

In another example of an evaluation protocol, the goal is also to identify edema or weight gain, but by identifying swelling of a patient's body part, such as the hands, face, or another body part. The patient interface device can provide instructions for the patient to position himself or herself a specific distance from the patient interface device, with the camera of the patient interface device pointing toward the patient. The instructions may direct the patient to hold his or her hands still in a specific position, such as placed on the surface where the patient interface device is positioned, in front of the patient interface device, with the thumbs near each other and the fingers spread, or the face held still. The instructions may direct the patient to hold the specified position for a period of time, such as ten seconds. The patient interface device can provide an illustration of the specified position and a timer counting down the period of time on the touch screen. The patient interface device can also show the video image being captured to the patient, so that the patient can make corrections in the position as needed. In one example, the patient interface device shows an outline of the desired body position, such as an outline of two hands, superimposed over the video actually being captured of the patient. The patient interface device captures and stores a video image of the patient's body part while the patient performs the evaluation protocol. Again, the system can provide the patient direct feedback on if the evaluation protocol was performed appropriately. The system can use facial recognition software or hand recognition software to identify the most relevant parts of the image and optimize the auto-focus and exposure time of the image capture process to optimize the image.

For both the first and second video portions, the system will identify an image within the video data for use in the pattern recognition analysis. The system can use facial recognition software or hand recognition software to identify the most relevant parts of the image. The pattern recognition analysis will quantify an area occupied by the patient's body part in each video image. Then the system will quantify the increase or decrease, if any, in the area occupied by the patient's body part from the first time to the second time on the image. One output of the analysis can be a statement of the quantification of the change, such as a statement that the patient's body part is swollen by 10% compared to the first time, or that there has been no increase in size. The system may compare the swelling to a threshold, and may provide information to the clinician based on that comparison. For example, the threshold may be that 5% or less swelling is of low concern, 5-20% is of some concern, and swelling of more than 20% is of significant concern. The report may state the level of concern based on the comparison of the patient's change to the threshold.

A third example of an evaluation protocol serves to analyze any tremor in a patient's body part, such as a hand. The patient interface device can provide instructions for the patient to position hold one hand a specific distance from the patient interface device, with the camera of the patient interface device pointing toward the patient. The instructions may direct the patient to hold his or her hand still in a specific position, such as with the palm facing the floor with the fingers spread. The instructions may direct the patient to hold the specified position for a period of time, such as one minute. The patient interface device can provide an illustration of the specified position and a timer counting down the period of time on the touch screen. The patient interface device can also show the video image being captured to the patient, so that the patient can make corrections in the position. In one example, the patient interface device shows an outline of the desired body position, superimposed over the video actually being captured of the patient. The patient interface device captures and stores a video image of the patient's body part while the patient performs the evaluation protocol.

For both the first and second video portions, the system will identify a video portion within the video data for use in the pattern recognition analysis. The pattern recognition analysis will quantify a level of tremor in each video portion. Then the system will quantify the increase or decrease, if any, in level of tremor displayed in the patient's body part from the first time to the second time on the video portion. One output of the analysis can be a statement of the quantification of the change, such as a statement that the patient's level of tremor increased by 10% compared to the first time, or that there has been no increase in a level of tremor. The system may compare the level of tremor to a threshold, and may provide information to the clinician based on that comparison. For example, the threshold may be that 5% or less increase in tremor is of low concern, 5-10% is of some concern, and an increase in level of tremor of more than 10% is of significant concern. The report may state the level of concern based on the comparison of the patient's change to the threshold.

In a fourth example of an evaluation protocol, the goal is to determine if the patient's breathing pattern has changed compared to a previous recording, such as by becoming more labored or shallow. In such an example, the patient interface device can provide instructions for the patient to position himself or herself a specific distance from the patient interface device, so that the patient's mouth is a specific distance from the microphone. The instructions may direct the patient to hold his or her breath for five seconds, and then breathe in a specific repetitive pattern, such as to take three slow, deep breaths. The instructions may indicate that the patient or the health care professional can touch a button on the touch screen when the patient has finished breathing in the specified pattern. A timer counting down the period of silence and the list of specific breathing patterns can be displayed on the touch screen. Again, the system can provide the patient direct feedback on if the evaluation protocol was performed appropriately.

The system will first identify useable portions within the audio/video data, for example starting two seconds into the period of not breathing and continuing until two seconds before the end button is activated. This identification step takes place for both the first and second audio/video data. The identified audio/video portions will be used for pattern recognition analysis and stored in the memory location. The pattern recognition analysis will compare the two portions of audio/video data and apply algorithms to determine if there are changes in the patient's breathing pattern. One output of the analysis can be a statement of the quantification of heart failure decompensation. For example, the report may state that the patient's breathing pattern shows respiratory distress. Breathing patterns are analyzed for both audio frequencies and for duration of inhalation and exhalation. The system may compare the change to a threshold, and may indicate if the patient's change exceeds the threshold. Thresholds can be established on an absolute basis or a relative percentage of change compared to prior data using an evaluation protocol.

### Clinician Location Systems and Methods

Returning to the example embodiment of FIG. 1, a clinician 118 at a remote location (the clinician location) interacts with a clinician interface device 120 to identify the patient, communicate with the patient 112, view IMD data and IMD settings, view audio/video data of the patient performing evaluation protocols and view pattern recognition analysis reports and outputs.

In the example illustrated in FIG. 1, the clinician interface device 120 has the same physical configuration as the patient interface device 116 and includes the same hardware and software features. In various embodiments, the clinician interface device 120 can include some or all of the features described herein with respect to the patient interface device 116.

It is also possible for the clinician interface device to take the form of an IMD programmer, a ruggedized laptop computer, a laptop computer, a desktop computer, a tablet computer, a smart phone or another configuration. The clinician interface device 120 can also be referred to as a device, programmer, user interface or external interface device. In one embodiment, the clinician interface device 120 is in communication with a server 132. The communication link between the clinician interface device 120 and the server 132 may be via phone lines, a wired network, the Internet 130, or any other data connection, such as a pervasive wireless network. The clinician interface device 120 can also be used when it is not in communication with an IMD device, but is only in communication with the server 132.

To identify the patient and verify patient identity, the clinician can view the identify data that is obtained by the patient interface device in one of the ways described herein.

### Further Options for Interface Device Hardware

The patient interface device 116 and clinician interface device 120 can each be referred to as an interface device. In some embodiments, an interface device is also a programmer of an IMD. As used herein, the term programmer refers to a device that programs IMDs and records data from IMDs. A programmer may also allow monitoring of the implanted device.

The interface device, also sometimes referred to as an external interface device, can display real-time data and/or stored data graphically, such as in charts or graphs, and textually through the user interface screen. In addition, the interface device can present textual information to a user along with several response options. The interface device can also input and store a user's response to a question, and can store a user's text response in some embodiments.

The interface device may be in communication with one or more servers having different purposes. For example, the interface device can communicate with a patient care server 132 where IMD data and patient data is stored. The interface device can also communicate with a server where electronic medical records are stored, which may be the patient care server 132 or another server. The communication link between the interface device and the one or more servers may be via phone lines, a pervasive wireless network, the Internet 130, or any other data connection.

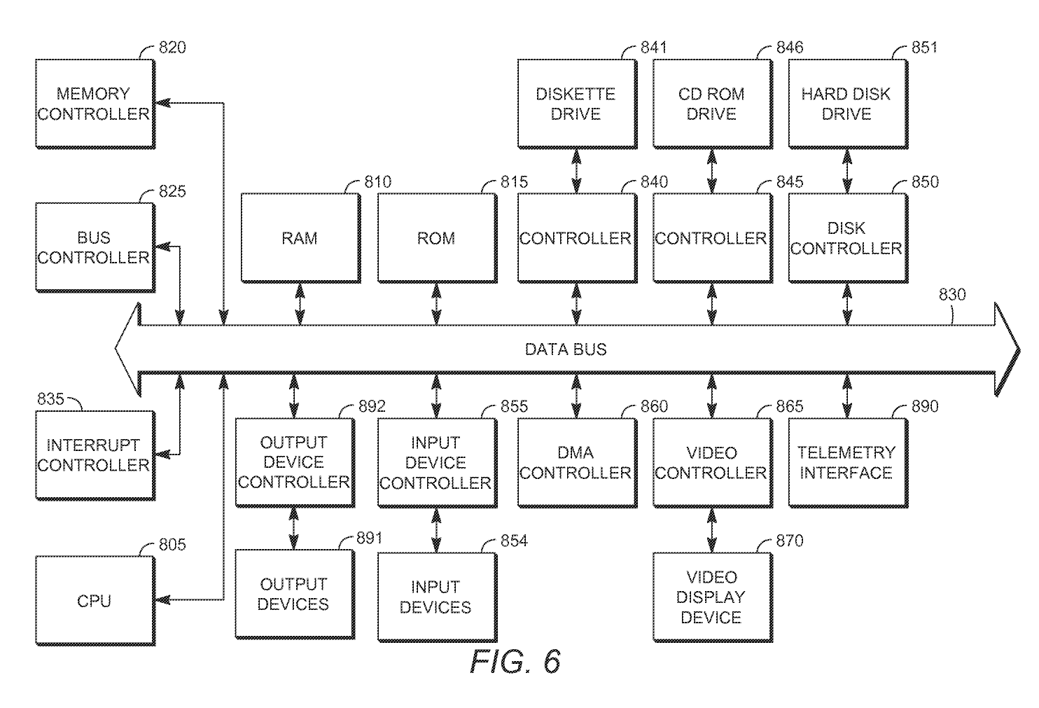

Interface devices can include components common to many computing devices, such as user interface devices to display and receive information from users. Referring now to FIG. 6, a diagram of various components of one embodiment of an interface device is shown in accordance with some embodiments of the invention. However, it is not required that an interface device or interface device have all of the components illustrated in FIG. 6, and an interface can include additional components.

In one embodiment, the interface device includes a central processing unit (CPU) 805 or processor, which may include a conventional microprocessor, random access memory (RAM) 810 for temporary storage of information, and read only memory (ROM) 815 for permanent storage of information. A memory controller 820 is provided for controlling system RAM 810. A bus controller 825 is provided for controlling data bus 830, and an interrupt controller 835 is used for receiving and processing various interrupt signals from the other system components.

Mass storage can be provided by diskette drive 841, which is connected to bus 830 by controller 840, CD-ROM drive 846, which is connected to bus 830 by controller 845, and hard disk drive 851, which is connected to bus 830 by controller 850. User input to the interface device system may be provided by a number of devices. For example, input devices 854 such as a keyboard and mouse can connect to bus 830 by input device controller 855. DMA controller 860 is provided for performing direct memory access to system RAM 810. A visual display is generated by a video controller 865 or video output, which controls video display 870. The system can also include a telemetry interface 890 or telemetry circuit which allows the system to interface and exchange data with an implantable medical device. In addition to the visual display, the interface device may include other components for communicating with a user, such as speakers. For example, output devices 891 such as speakers can connect to bus 830 by output device controller 892.

A pattern recognition analysis module, a seeding module, parameter interaction module, pace timing optimization module and combinations thereof can be present in the interface device in different embodiments.

### Hardware Options for IMDs

For convenience, the description herein will occasionally make reference to cardiac devices and issues with treating tachyarrhythmia. However, the methods and systems described herein are broadly applicable to many different types of IMDs, including non-cardiac devices. The applications discussed herein are intended to be exemplary and not limiting.

One embodiment of an IMD system will now be described in more detail with reference to FIG. 1, which is a schematic of an exemplary telemedicine system including an IMD. The system can include an implantable medical device 110 disposed within a patient 112. The implantable medical device 110 can be in communication with an interface device 116. In some embodiments, communication between the implantable medical device 110 and the interface device can be via inductive communication through a telemetry wand 128 held on the outside of the patient 112 near the implantable medical device 110. However, in other embodiments, communication can be carried out via radiofrequency transmission, acoustically, or the like.

The implantable medical device 110 can include one or more implantable sensors in order to gather data regarding the patient 112. For example, the implantable medical device 110 can include an activity level sensor, a respiration sensor, a blood pressure sensor, an impedance sensor, or other sensors.

The implantable medical device 110 can be configured to store data over a period of time, and periodically communicate with the interface device 116 or another device in order to transmit some or all of the stored data.

In one embodiment, the IMD 110 is a CRM device and is coupled by one or more leads 122 to the heart 126. Cardiac leads 122 include a proximal end that is coupled to IMD 110 and a distal end, coupled by an electrode or electrodes to one or more portions of a heart 126. The electrodes typically deliver cardioversion, defibrillation, pacing, or resynchronization therapy, or combinations thereof to at least one chamber of the heart 126. The electrodes may be electrically coupled to sense amplifiers to sense electrical cardiac signals.

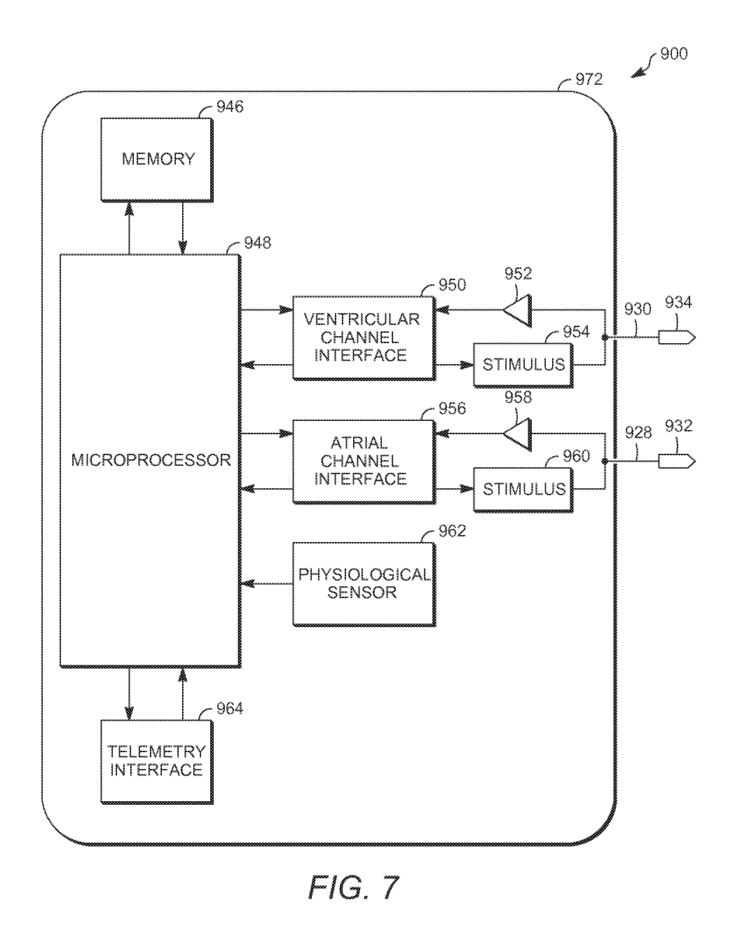

Referring now to FIG. 7, some components of an exemplary implantable system 900 are schematically illustrated. The implantable medical system 900 can include an implantable medical device 972 coupled to one or more stimulation leads 930 and 928. The implantable device 972 can also include one or more physiological sensors 962, or other sensors, such as a pressure sensor, impedance sensor and others.

The implantable device can include a microprocessor 948 (or processor) that communicates with a memory 946 via a bidirectional data bus. The memory 946 typically comprises ROM or RAM for program storage and RAM for data storage. The implantable device can be configured to execute various operations such as processing of signals and execution of methods as described herein. A telemetry interface 964 is also provided for communicating with an external unit, such as a interface device or a patient management system.

The implantable device can include ventricular sensing and pacing channels comprising sensing amplifier 952, output circuit 954, and a ventricular channel interface 950 which communicates bi-directionally with a port of microprocessor 948. The ventricular sensing and pacing channel can be in communication with stimulation lead 930 and electrode 934. The implantable device can include atrial sensing and pacing channels comprising sensing amplifier 958, output circuit 960, and an atrial channel interface 956 which communicates bi-directionally with a port of microprocessor 948. The atrial sensing and pacing channel can be in communication with stimulation lead 928 and electrode 932. For each channel, the same lead and electrode can be used for both sensing and pacing. The channel interfaces 950 and 956 can include analog-to-digital converters for digitizing sensing signal inputs from the sensing amplifiers and registers which can be written to by the microprocessor in order to output pacing pulses, change the pacing pulse amplitude, and adjust the gain and threshold values for the sensing amplifiers.

A seeding module, parameter interaction module, pace timing optimization module and combinations thereof can be present in the device in different embodiments.

It should be noted that, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as “arranged”, “arranged and configured”, “constructed and arranged”, “constructed”, “manufactured and arranged”, and the like.

One of ordinary skill in the art will understand that the modules, circuitry, and methods shown and described herein with regard to various embodiments of the invention can be implemented using software, hardware, and combinations of software and hardware. As such, the illustrated and/or described modules and circuitry are intended to encompass software implementations, hardware implementations, and software and hardware implementations.

All publications and patent applications in this specification are indicative of the level of ordinary skill in the art to which this invention pertains. All publications and patent applications are herein incorporated by reference to the same extent as if each individual publication or patent application was specifically and individually indicated by reference.

This application is intended to cover adaptations or variations of the present subject matter. It is to be understood that the above description is intended to be illustrative, and not restrictive. The scope of the present subject matter should be determined with reference to the appended claims, along with the full scope of equivalents to which such claims are entitled.

What is claimed is:

1. A method for assisting a remote implantable medical device (IMD) clinician with evaluation of a patient having an IMD, comprising:
a. capturing audio/video data of the patient while the patient performs a first evaluation protocol at a first location;
b. performing a pattern recognition analysis of the audio/video data of the patient, comprising comparing specific portions of the audio/video data to like portions of previous audio/video data of the patient that were recorded while the patient performed the first evaluation protocol; and
c. providing a report of any changes identified during the pattern recognition analysis.

2. The method of claim 1 further comprising sending the report to an electronic medical record, an additional interface device, or both.

3. The method of claim 1 wherein the report includes IMD sensor data.

4. The method of claim 1 wherein the report includes at least one of the group consisting of changes in the patient's weight, changes in the patient's pulse oximetry and changes in the patient's blood pressure.

5. The method of claim 1 further comprising generating an alert if the changes identified during the pattern recognition analysis meet alert threshold requirements.

6. The method of claim 1 wherein the report includes a heart failure decompensation status of the patient.

7. The method of claim 1 further comprising the step of communicating with the IMD to identify the IMD or patient, wherein the IMD stores identity data that uniquely identifies the IMD or the patient having the IMD.

8. The method of claim 7 wherein the step of identifying the IMD or patient comprises inductive communication or radio frequency communication between a patient interface device and the IMD.

9. The method of claim 1 wherein the capturing step comprises at least one of the following group:
a. capturing audio data of the patient's speech while the patient repeats specific words to determine if there has been any frequency shift in the patient's voice;
b. capturing an image of a body part of the patient while the patient holds the body part in a specified position to determine if volume of body part has increased;
c. capturing video data of a body part of the patient while the patient holds the body part in a specified position to determine if patient has a tremor condition; and
d. capturing audio/video data of a patient breathing in a specific pattern to determine a breathing rate and pattern.

10. The method of claim 1 further comprising identifying data portions of the audio/video data for analysis.

11. The method of claim 1 further comprising:
a. providing a patient interface device at a patient location configured to retrieve identity data and sensor data from an implantable medical device (IMD);
b. providing a clinician interface device at a clinician location configured to be in remote, bi-directional audio/video communication with the patient interface device;
c. wherein the patient interface device captures the audio/video data of the patient and wherein the report is provided to the clinician interface device.

12. A method for assisting a remote implantable medical device (IMD) clinician with evaluation of a patient having an IMD, comprising:
a. providing a patient interface device at a patient location configured to retrieve identity data and sensor data from an implantable medical device (IMD), wherein the patient interface device is further configured to receive IMD data from the IMD;
b. providing a clinician interface device at a clinician location configured to be in remote, bi-directional audio/video communication with the patient interface device;
c. the patient interface device capturing audio/video data of the patient while the patient performs a first evaluation protocol at the first location; and
d. performing a pattern recognition analysis of the audio/video data of the patient, comprising comparing specific portions of the audio/video data to like portions of previous audio/video data of the patient that were recorded while the patient performed the first evaluation protocol; and
e. providing to the clinician interface device a report of any changes identified during the pattern recognition analysis; and
f. generating an alert if the changes identified during the pattern recognition analysis meet alert threshold requirements.

13. A system for assisting a remote implantable medical device (IMD) clinician with evaluation of a patient having an IMD, comprising:
a. a patient interface device configured to:
i. retrieve identity data and sensor data from an implantable medical device (IMD) at a first location, and
ii. capture audio/video data of the patient while the patient performs a first evaluation protocol at the first location;
b. a clinician interface device configured to be in remote, bi-directional audio/video communication with the patient interface device;
c. wherein the system is configured to perform a pattern recognition analysis of the audio/video data of the patient, comprising comparing specific portions of the audio/video data to like portions of previous audio/video data of the patient that were recorded while the patient performed the first evaluation protocol; and
d. wherein the system is configured to provide to the clinician interface device a report of any changes identified during the pattern recognition analysis.

14. The system of claim 13 further comprising an alert module configured to generate an alert if the changes identified during the pattern recognition analysis meet alert threshold requirements.

15. The system of claim 13 wherein the patient interface device further comprises:
a first communication module configured to communicate with the clinician interface device; and
a device communication module configured to communicate with the IMD, retrieve identity data from the IMD and program the IMD with IMD settings.

16. The system of claim 15 wherein the first communication module of the patient interface device is configured for communication with the clinician interface device over one of the Internet or a wired network.

17. The system of claim 15 wherein the device communication module of the patient interface device is configured for one of inductive communication and radio frequency communication with the IMD.

18. The system of claim 13 wherein the capturing step comprises at least one of the following group:
a. capturing audio data of the patient's speech while the patient repeats specific words to determine if there has been any frequency shift in the patient's voice;
b. capturing an image of a body part of the patient while the patient holds the body part in a specified position to determine if volume of body part has increased;
c. capturing video data of a body part of the patient while the patient holds the body part in a specified position to determine if patient has a tremor condition; and
d. capturing audio/video data of a patient breathing in a specific pattern to determine a breathing rate and pattern.

19. The system of claim 13 wherein the report includes a heart failure decompensation status of the patient.

20. The system of claim 13 wherein the patient interface device is configured to program the IMD with IMD settings.
